(19) United States
(12) Patent Application Publication
(10) Pub. No.: US 2009/0131744 A1
(43) Pub. Date: May 21, 2009

(54) Sexual Aid Device

(76) Inventor: John Maurice Pattenden, Mystic, CT (US)

Correspondence Address: John Pattenden, 199 Heather Glen Lane, Mystic, CT, 06355, US

(21) Appl. No.: 12/125,031

(22) Filed: May 21, 2008

Related U.S. Application Data
(60) Provisional application No. 60/940,111, filed on May 25, 2007.

(51) Int. Cl. A61F 5/00

(52) U.S. Cl. 600/38

(57) ABSTRACT

Disclosed is a sexual aid device comprising an inner region, and an outer region, wherein at least the outer region is fabricated of a molded, lofted foam material that is non-porous, wherein the outer region is configured for use with at least one of an orifice, genitalia and erogenous zone of at least one user.

### CROSS REFERENCE TO RELATED APPLICATIONS

This application claims the benefit of PPA Ser. No. 60/940,111, filed May 25, 2007 by the present inventor, which is incorporated by reference.

### FEDERALLY SPONSORED RESEARCH

Not applicable

### SEQUENCE LISTING OR PROGRAM

Not applicable

### FIELD OF THE INVENTION

The disclosure relates generally to a sexual aid device, and more particularly to a sexual aid device at least partially fabricated of a molded, lofted foam material that is non-porous.

### BACKGROUND OF THE INVENTION

Sexual aid devices have been used to enhance sexual experiences throughout time. These devices include dildos, vibrators, strap-ons, anal plugs, vaginal/anal beads, penile rings, Ben-Wa-Balls, etc. In modern times, with the development of plastics and other resilient materials, sexual devices have been mass produced in a wide variety of forms that are both pleasing in shape and design.

However, many sexual aid devices of the prior art are difficult to sterilize because they cannot be cleaned at very high temperature without melting or deforming. In addition, many of these prior art devices retain odor, bacteria and fungi because they are somewhat porous. Moreover, many of the devices of the prior art do not satisfactorily adjust quickly to, or hold, body temperatures during sex acts. In addition, many of the devices used in the prior art comprise materials that can be unsafe and possibly toxic in nature. Silicone devices have issues as well. One such issue is silicone's inability to be used with silicone lubricate because this causes silicone to melt. In addition, high-grade silicone is very expensive. For example, a device using FDA approved silicone would be estimated to retail in excess of $750. Further, many devices labeled as being manufactured from silicone may only contain a small percentage of the material to be allowed to make such a claim (it is only necessary for a device to comprise of ten percent or more for it to be labeled as a silicone product). It is also noted that there are now devices being made from thermoplastic elastomers (TPE), but devices made from TPE can include issues in that they can be at least somewhat porous.

Furthermore, because the phallic portion and base plate portion of a strap-on are not desirably made of the same material, a strap-on must comprise a system of releasable/detachable parts or separate parts bonded/welded together. Multiple part systems such as this can lead to joints and crevices that are difficult to impossible to clean, which can make for an unsanitary device. These multiple part systems (such as an O-ring design) can also be prone to undesirable movement and uncomfortable against a user's skin.

Therefore, it can be appreciated that there exists a continuing need for new and improved sex aid devices that can be used for increasing stimulation during sex acts offering comfort, while also being anti-microbial (i.e. resisting odor and inhibiting bacterial and fungal growth), non-toxic, easily sanitized, and capable of quickly adjusting to and retaining of body temperature.

### SUMMARY OF THE INVENTION

Disclosed is a sexual aid device comprising an inner region, and an outer region, wherein at least the outer region is fabricated of a molded, lofted foam material that is non-porous, wherein the outer region is configured for use with at least one of an orifice, genitalia and erogenous zone of at least one user.

### BRIEF DESCRIPTION OF THE FIGURES

FIG. 1a illustrates a front perspective view of first exemplary embodiment of a sexual aid device;

FIG. 1b illustrates a side perspective view of the first exemplary embodiment the sexual aid device;

FIG. 1c illustrates a top perspective view of the first exemplary embodiment the sexual aid device;

FIG. 1d illustrates a front perspective view of the first exemplary embodiment the sexual aid device without a piece configured for sexual pleasure;

FIG. 2 illustrates a side perspective view of second exemplary embodiment of a sexual aid device;

FIG. 3a illustrates a side perspective view of third exemplary embodiment of a sexual aid device;

FIG. 3b illustrates a side perspective view of fourth exemplary embodiment of a sexual aid device;

FIG. 4 illustrates a side perspective view of fifth exemplary embodiment of a sexual aid device;

FIG. 5 illustrates a side perspective view of sixth exemplary embodiment of a sexual aid device;

FIG. 6 illustrates a side perspective view of seventh exemplary embodiment of a sexual aid device;

FIG. 7 illustrates a side perspective view of eighth exemplary embodiment of a sexual aid device;

FIG. 8 illustrates a side perspective view of ninth exemplary embodiment of a sexual aid device;

FIG. 9a illustrates a sexual aid device mold in accordance with various embodiments of the present invention;

FIG. 9b illustrates a cut away cross section of a sexual aid mold;

FIG. 9c illustrates top view of the interior of the lower portion of the mold.

### DETAILED DESCRIPTION

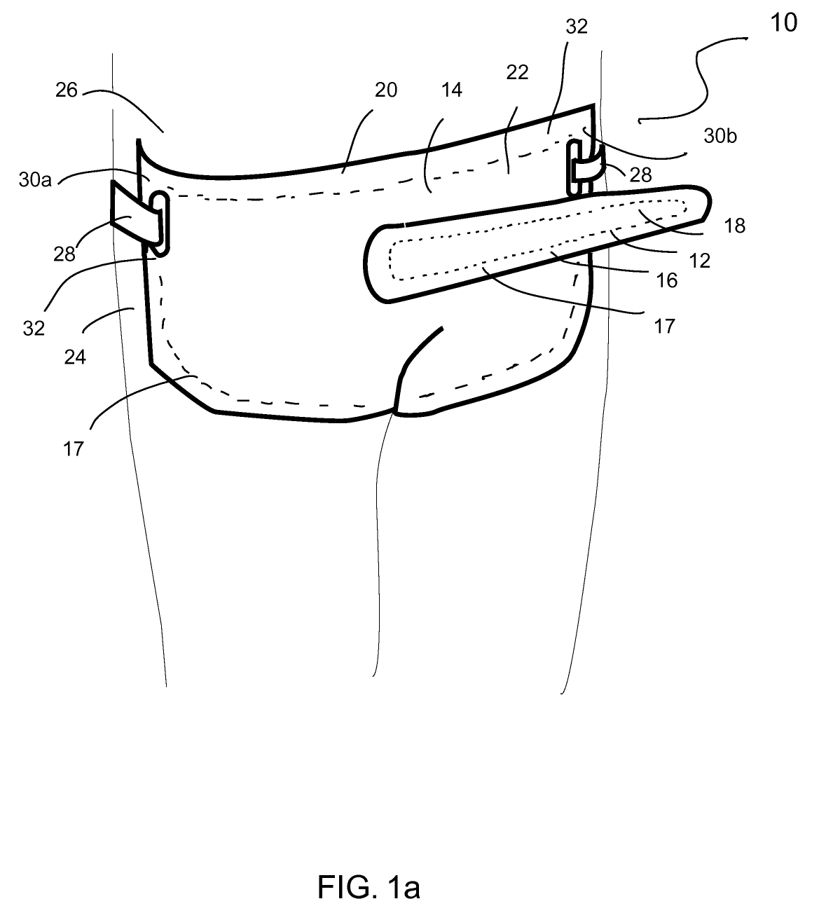

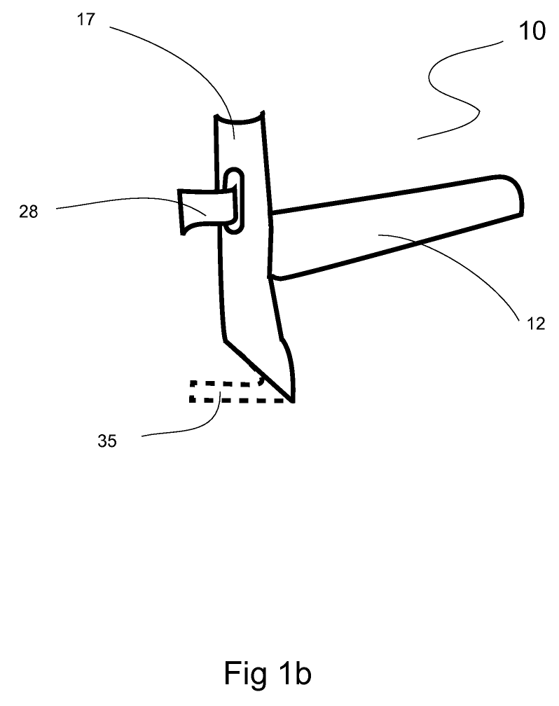

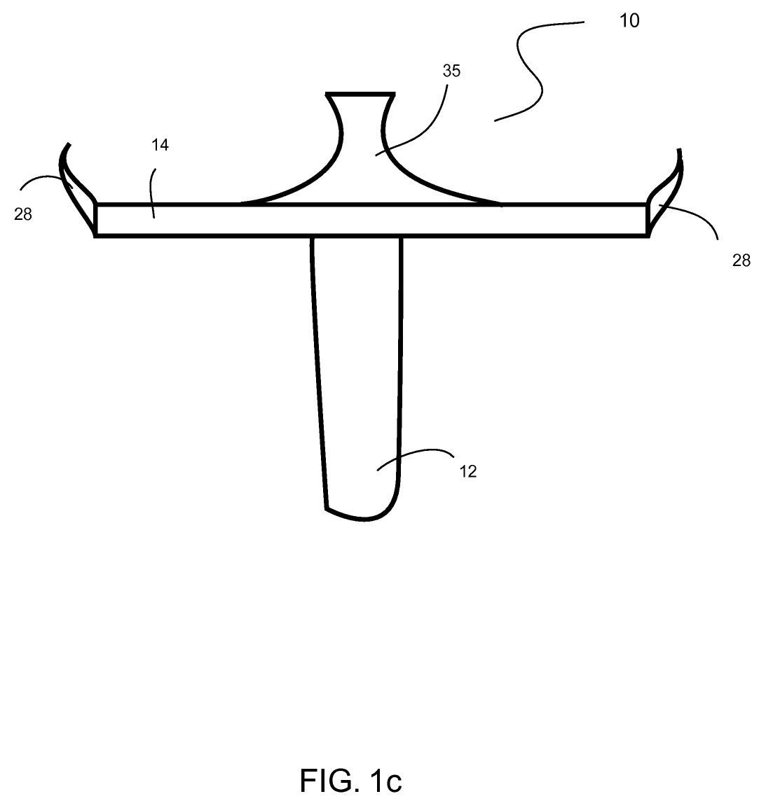

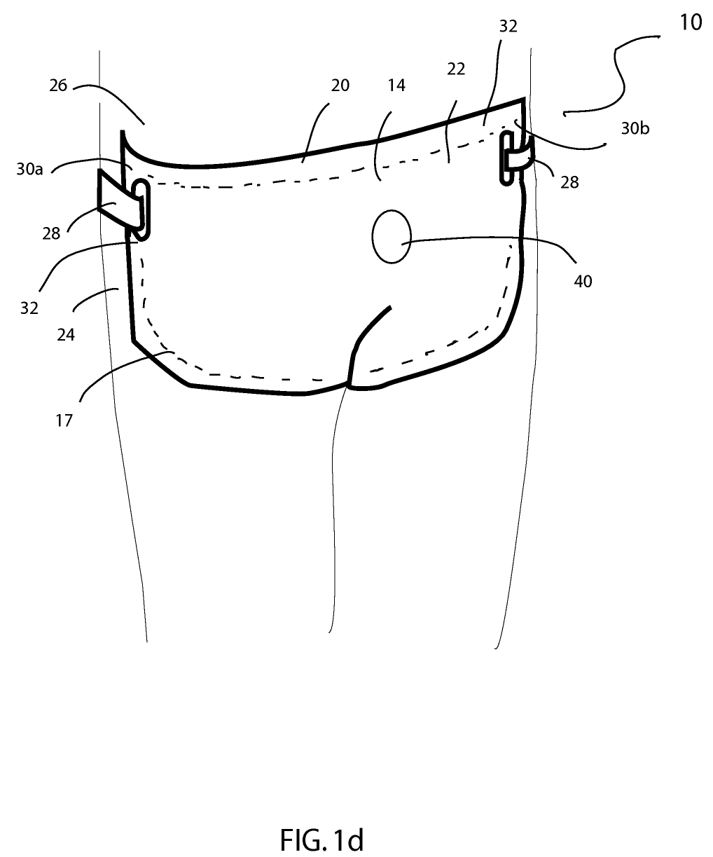

Referring to FIGS. 1a-c, an exemplary embodiment of sexual aid device 10 is illustrated. In the embodiment of FIGS. 1a-c, the aid device 10 is a “strap-on” device 10, referred to hereinafter as the device 10. The device 10 includes a piece configured for sexual pleasure 12 (such as a phallic shaped piece) and a harness or base plate 14 that includes a crotch cup 35. The piece 12 includes an outer region 16 that is fabricated of a molded lofted foam material 17 that is a non-porous, non-toxic, closed cell polymer resin (CCPR) material, which it is neither plastic, nor rubber. The piece 12 also includes an inner region 18, which may or may not be fabricated of the molded, lofted foam material 17 that is non-porous. Similarly, the base plate 14 includes an outer base region 20 that is fabricated of the molded, lofted foam material 17 that is non-porous and an inner base region 22, which may or may not be fabricated of the molded, lofted foam material 17 that is non-porous. At least the outer region 16 of the piece 12 and the inner region of the outer base region 20 are molded together in a one-piece, non-releasable formation. However, it should be appreciated that the inner region 18, the outer region 16, the inner base region 22, and the outer base region 20 may all be fabricated of the same molded, lofted foam material that is non-porous, wherein each of these regions are in molded, non-releasable formation with each other.

As is shown in the Figures, and in particularly FIG. 1a, the base plate 14 is configured to fit snuggly against a lower torso region 24 of a user 26. That is, the base plate 14 includes a general concavity that matches the contour of the groin and hip region of the user 26. The base plate 14, and by extension the piece 12, are affixed to the user 26 via straps 28. These straps 28 extend from opposite first and second lateral ends 30a and 30b of the base plate 14. The straps 28 may be of the same molded, lofted foam material 17 as the base plate 14, and associate with each other via a belt buckle, VELCRO™ other fastening means disposed at relative ends of the straps. The straps 28 may also be of separate construction with the base plate 14, and associated with the base plate 14 via strap cavities 32. If the straps 28 are of separate construction from the base plate 14, they may comprise any desirable material which acts as a belt (possibly a single belt), including but not limited to the molded, lofted foam material 17 of the base plate 14, leather, latex, rubber, or VELCRO™.

It should be noted that because the piece 12 and base plate 14 are in molded, non-releasable formation with each other, there are no joints or crevices between the two pieces in which contaminating bodily fluid or discharge can be trapped. The crotch cup 35 also eliminates the need for belts or straps that run under a user's crotch. In addition, because of the non-porous nature of the lofted foam material 17, the device 10 is resistant to the microbes and fungi that can cause odor and infection. The foam material 17 is also easily sterilized.

As shown in FIG. 1d, it should also be appreciated that the base plate 14 may be configured without the piece 12, wherein the base plate comprises the molded, lofted foam material that is non-porous discussed above, and is further configured for association with a separate dildo (such as the one described below) or other sexual aid device. The base plate 14 may be configured for this type of association via a holding opening 40 defined at a portion of the base plate 14 desirable for dildo location. The dildo may be inserted into the holding opening 40 (which may be adjustable), and held in place via, but not limited to, a screw or a bayonet or a frictional fit, and/or flange located at an end of the dildo closest to a user's body. This type of association with a separate dildo offers a user greater control, increased comfort, a more sanitary and snug fit between the dildo and base plate 14, than is typically found in prior art designs such as an O-ring.

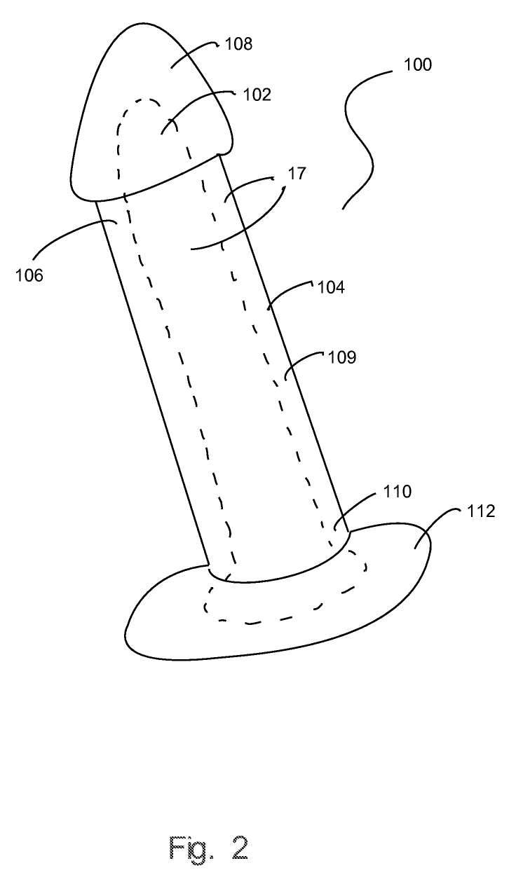

Referring now to FIGS. 2-8, multiple embodiments of a sexual aid device are illustrated. As is discussed above, devices or device components that are fabricated of the lofted foam material 17 is closed cell in nature, anti-microbial (which resists odor and inhibits bacterial and fungal growth), and non-toxic. The non-porous nature of this material also lends itself to improved lubricant retention. Each of the multiple embodiments will now be discussed in turn.

Referring to the exemplary embodiment of FIG. 2, a sexual aid device in the form of a dildo 100 is illustrated. The dildo 100 includes an inner region 102 and an outer region 104. It should be appreciated that though the dildo 100 is illustrated to be entirely fabricated of the lofted foam material 17 discussed above, it may also include an inner region 102 of a different material.

The dildo 100 of FIG. 2 includes a phallic shaped body 106, though the body 106 may be a phallic, finger, or other shape configured to enhance sexual pleasure. The body 106 includes a head end 108, a shaft 109, and a base end 110. A flange portion 112, which may be cupped for suction purposes, extends from the base end 110. Each of the head end 108, shaft 109, base end 110, and flange portion 112 are in molded, non-releasable formation with each other.

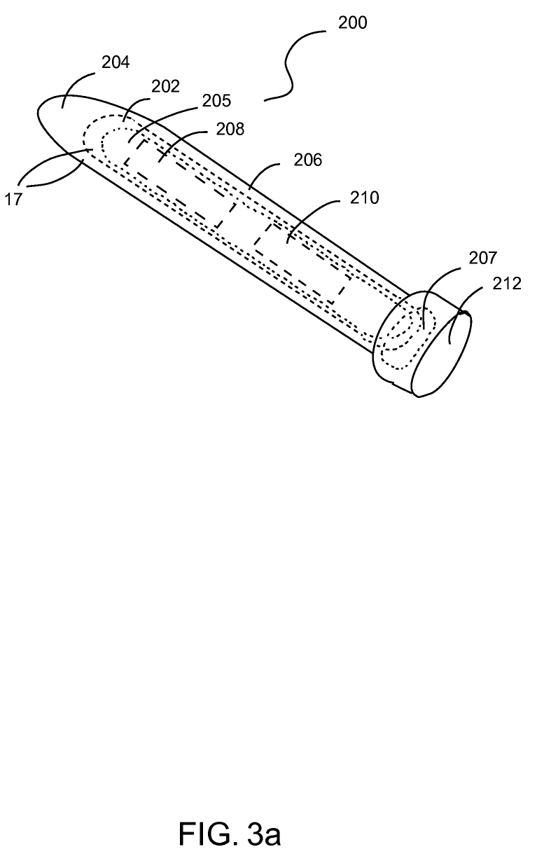

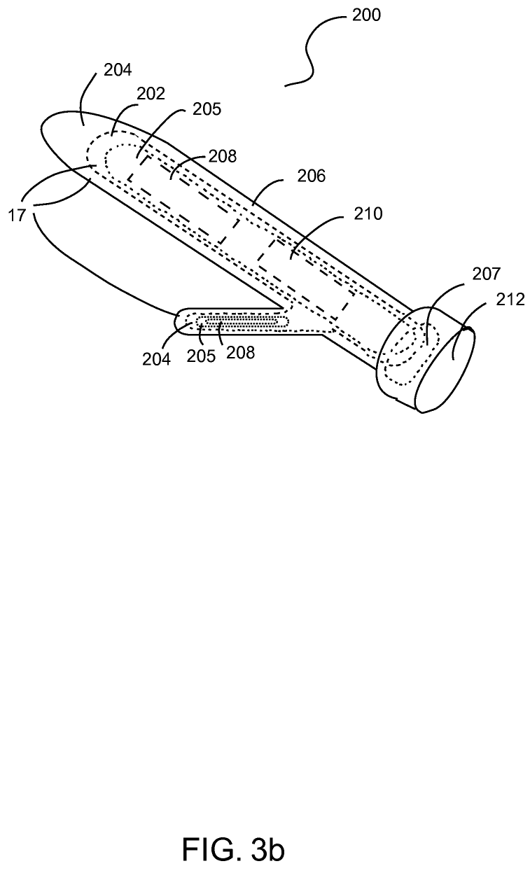

Referring to the exemplary embodiment of FIGS. 3a and b, a sexual aid device in the form of a vibrator 200 is illustrated. The vibrator 200 includes an inner region 202 and an outer region 204, with the inner region defining a cavity 205. It should be appreciated that though the inner region 202 and the outer region 204 are illustrated to be entirely fabricated of the lofted foam material 17 discussed above, the inner region 202 may also be fabricated of a different material.

The vibrator of FIG. 3a includes a phallic shaped body 206, similar to that of the dildo 100, though the body 206 may be a phallic, finger, or other shape configured to enhance sexual pleasure. As is illustrated in the Figure, the cavity 205 opens at one end of the body 206, so as to allow an entrance 207 to the cavity 205. An agitator 208 and a power-providing device 210, which may be disposed within the cavity 205 via the entrance 207, are also illustrated. In an exemplary embodiment, the power-providing device 210 is a battery in electric association with a motorized agitator 208. It should be appreciated that the power providing device 210 and controller for speed may be disposed out of the cavity 205, and electrically associated with the agitator 208 via a wire that extends from the cavity 205. Power can be supplied by batteries, re-chargeable batteries or plugged into a wall outlet. A detachable cap 212 is used to cover the entrance 207, and may be used to activate and vary the speed of the agitator 208 in a conventional vibrator activating manner (i.e. via twisting of the cap 212 which is operably coupled to the battery).

The FIG. 3b is an embodiment of a vibrator that includes a furcate that is intended to vibrate against the clitoral and external vaginal area.

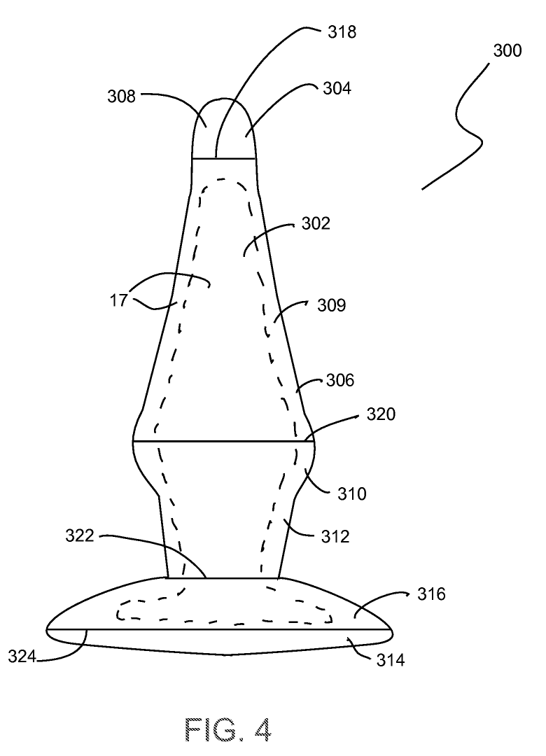

Referring to the exemplary embodiment of FIG. 4, a sexual aid device in the form of an anal plug 300 is illustrated. The anal plug 300 includes an inner region 302 and an outer region 304. It should be appreciated that though the anal plug 300 is illustrated to be entirely fabricated of the lofted foam material 17 discussed above, it may also include an inner region 302 of a different material.

As shown in FIG. 4, the anal plug 300 includes a device body 306. The body 306 includes a bulbous conical end 308, an enlarging region 309, a cylindrical portion 310, a decreasing portion 312, a flange end 314, and a flange 316. The enlarging region 309 extends from the conical end 308 to the cylindrical portion 310, while the decreasing portion 312 extends from the cylindrical portion 310 to the flange end 314. As is shown in FIG. 4, the enlarging region 309 increases in diameter as it extends from a smaller first diameter 318 at the conical end 308 to a larger second diameter 320 of the cylindrical portion 310. In contrast, the decreasing region 312 increases in diameter as it extends from the larger second diameter 320 at the cylindrical portion 310 to a smaller third diameter 322 of the flange end 314. As is also shown in FIG. 4, the flange 316 includes a fourth and largest diameter 324, and is in molded, non-releasable formation with the flange end 314 of the body 306.

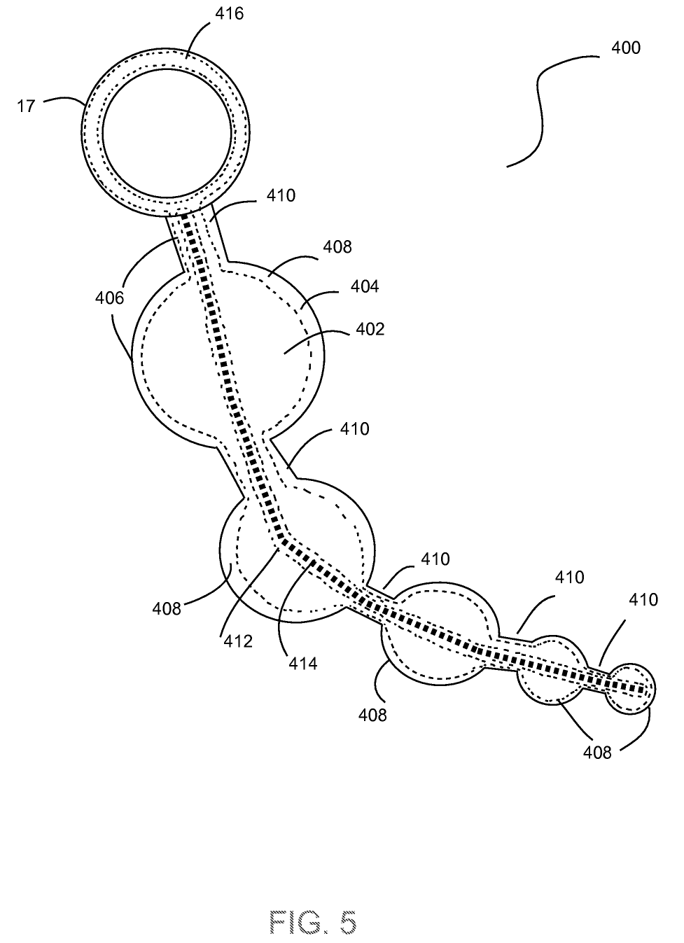

Referring to the exemplary embodiment of FIG. 5, a sexual aid device in the form of a vaginal/anal beads 400 is illustrated. The vaginal/anal beads 400 include an inner region 402 and an outer region 404. It should be appreciated that though the inner region 402 and the outer region 404 are illustrated to be entirely fabricated of the lofted foam material 17 discussed above, the inner region 402 may also be fabricated of a different material.

As shown in the Figure, the vaginal/anal beads 400 include a device body 406 that comprises a plurality of ball shaped regions 408 connected via connecting portions 410. These connecting regions 410 may be solely responsible for connecting the ball regions 408, or, as shown in FIG. 5, the inner region 402 of the body 406 may define a string cavity 412 that runs a substantial length of the body 406, and be configured to hold a string component 414 (such as fishing line) that aids the connecting regions 410 in holding the ball regions 408 together. As is further shown in FIG. 5, the ball regions 408 of the body 406 may be of varying sizes (increasing/decreasing from one end of the body 408 to the other), and a pull ring 416 (fabricated of the lofted foam material 17) may be disposed at one end of the body 406 in non-releasable formation that end of the body 406.

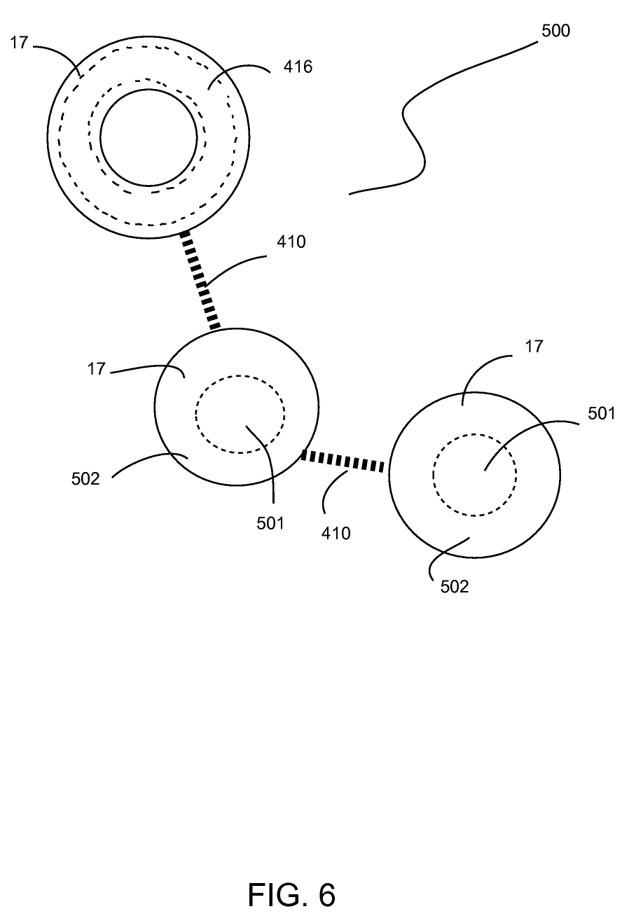

Referring to the exemplary embodiment of FIG. 6, a sexual aid device in the form of covered Ben-Wa-Balls 500 is illustrated. Ben-Wa-Balls 501 associated with the covered Ben-Wa-Balls 500 are encased in a coating 502 of lofted foam material 17. Like the vaginal/anal beads 400, the coated Ben-Wa-Balls 500 are associated via connecting portion 410 that may each define a string cavity 412 for holding a connection strengthening string component 414. The Ben-Wa-Balls also include a pull ring 416.

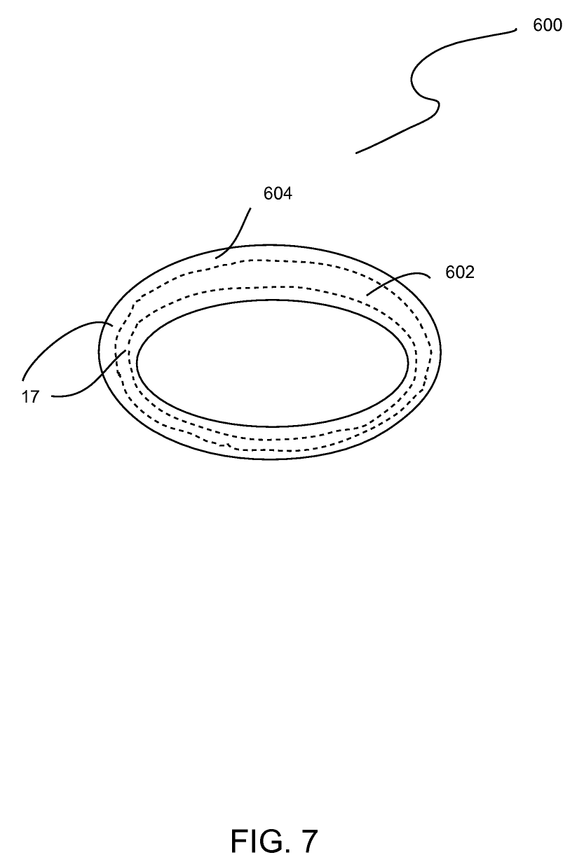

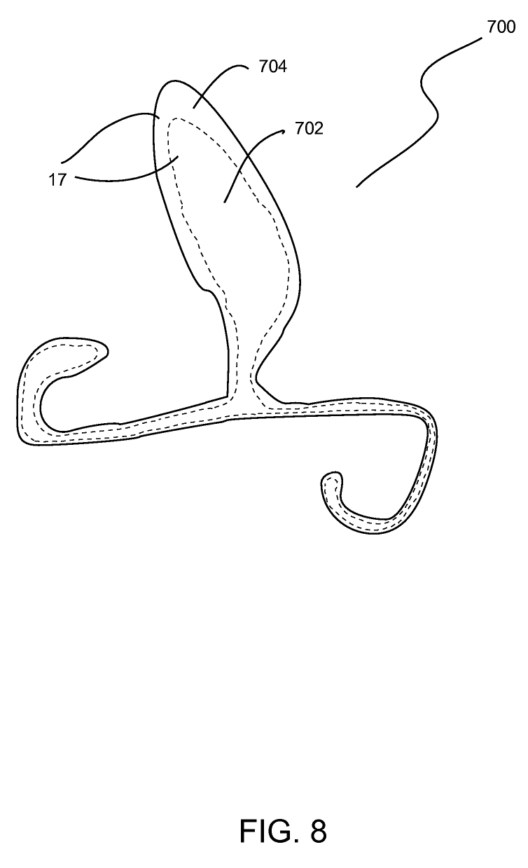

Referring to the exemplary embodiments of FIGS. 7 and 8 a sexual aid devices in the form of a penile ring 600 (FIG. 7) and anal prostate dildo 700 (FIG. 8) are illustrated. Both the penile ring 600 and anal prostate dildo 700 include inner region 602 and 702 and outer regions 604 and 704. It should be appreciated that though the anal penile ring 600 and anal prostate dildo 700 are illustrated to be entirely fabricated of the lofted foam material 17 discussed above, they may also include inner regions 602 and 702 of different materials.

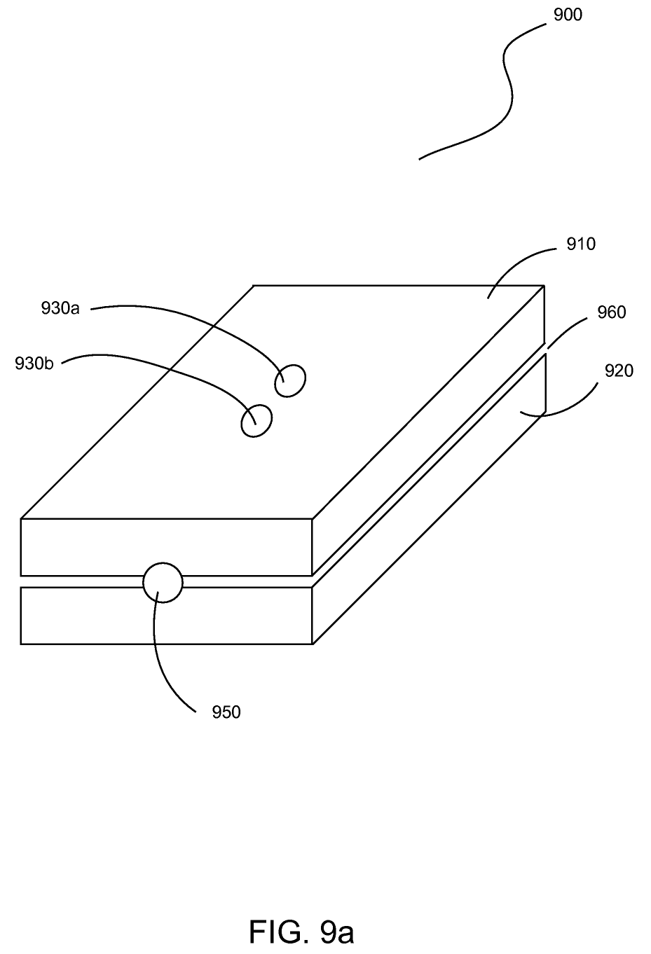

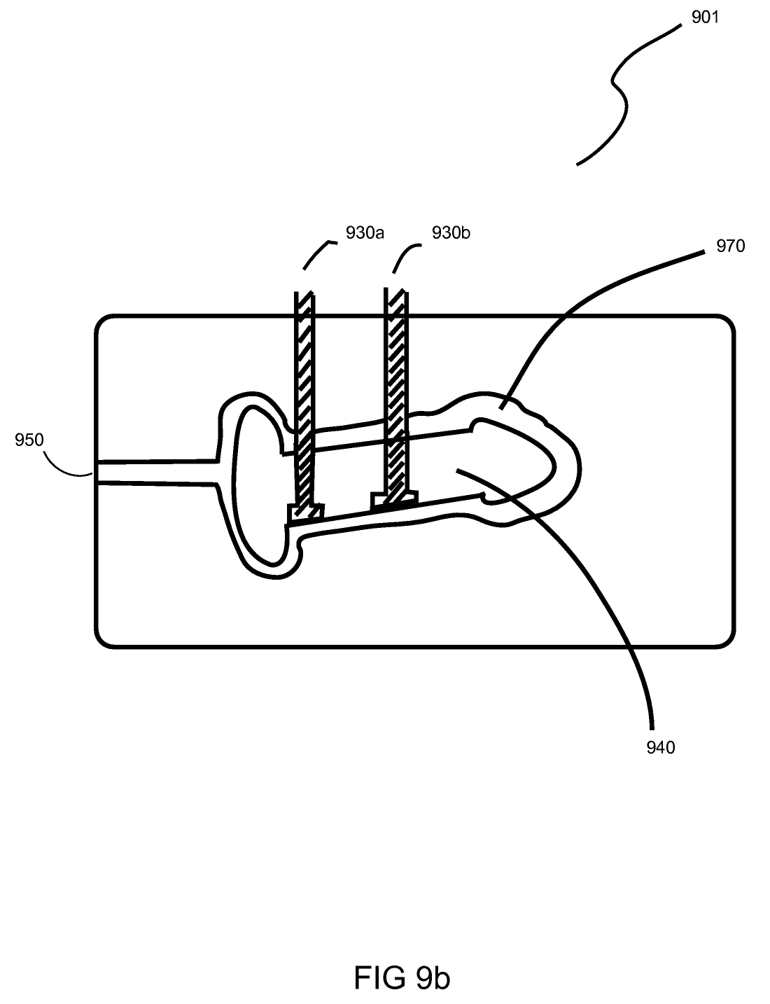

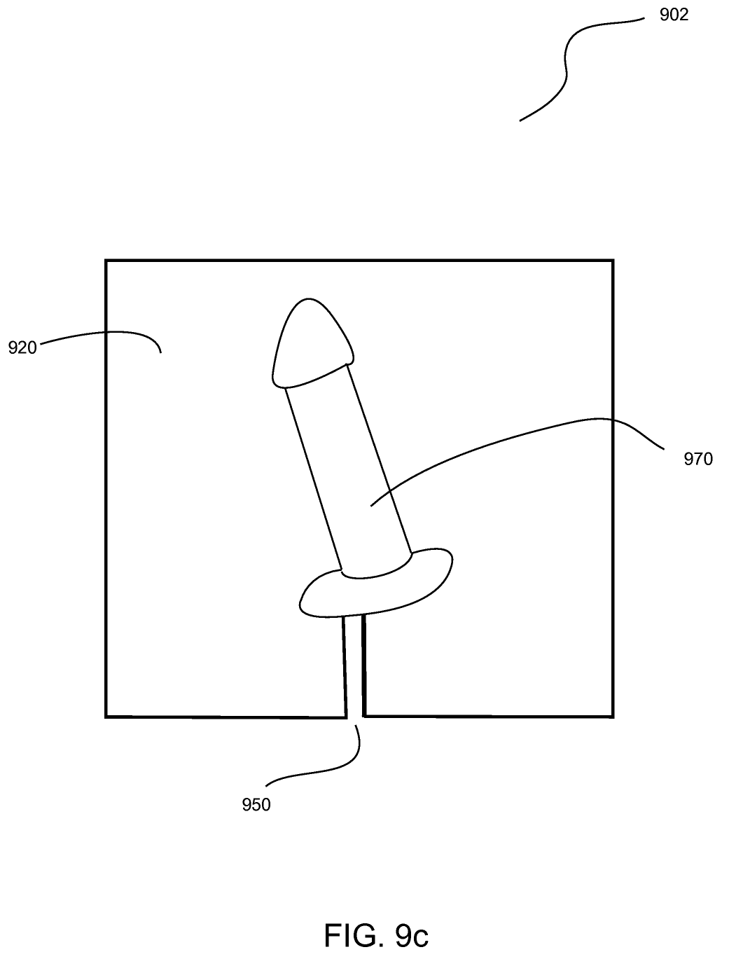

It should be appreciated that each instance of the lofted foam material 17 discussed above may (in an exemplary embodiment) specifically comprise a closed cell resin known as CROSLITE™. CROSLITE™ is an extraordinary impact absorbing resin material developed for maximum cushioning. Its closed cell properties resist odor, inhibits bacterial and fungal growth and are nontoxic. This versatile material can be worn next to skin and be cleaned with just antibacterial soap and water or sterilized in boiling water.” This CROSLITE™ resin may include a pre-mixture of resin, pigment, and a growth additive. As shown in FIGS. 9a,b, and c, the sexual aid devices discussed above may be molded as will be discussed herein. The resin of the CROSLITE™ material or other suitably non-porous lofted foam material 17, originally in pellet form, is heated to a liquid state. This liquid resin is screwed into a mold that has been heated prior to receiving the resin. The volume of resin screwed into the mold is controlled by the pitch of the screw that drives the liquid resin into the mold. The liquid resin is allowed to set, at which time the mold is opened and the sexual aid device is removed from the mold. The formed sexual aid device piece is then placed on a cooling last, where it is allowed to air dry.

During this process, a relatively small sexual aid device conforming to the size of the mold is created, but when the mold opens, the sexual aid device springs out as it expands in size. Then, as the sexual aid device is air cooled, it contracts to a final size. Thus, the process involves both an expansion characteristic and a contraction characteristic. Multiplying the size of the sexual aid device piece in the mold by the expansion characteristic yields the size of the sexual aid device after the mold is opened. Multiplying the expansion characteristic by the contraction characteristic provides a final growth value representative of the final size of the sexual aid device relative to the mold.

In such a manufacturing process, a number of elements can be controlled to achieve the desired end result. These elements include, the volume of material introduced into the mold, the size of the mold, the composition of the material being used, and the size of the cooling last. Embodiments disclosed herein use a fixed volume and composition of material, and cooling last size, while varying mold sizes to control the size of the end product. This approach is particularly valuable for sexual aid devices manufactured of the same color resin. Where different colors are involved, the composition of the resin may be varied across the colors to achieve size control between colors. This composition adjustment is more fully described below.

In one particular embodiment, the resin is Ethylene Vinyl Acetate copolymer (EVA) based material. In this particular case, additives are included with the EVA base to create an expansible and cross-linking material. More particularly, an expanding powder is added which decomposes at a specific temperature to produce gases which cause the material to rise as it sets within a mold. Accordingly, when the mold is opened, an instantaneous expansion of the molded part results. During this expansion, the dimensions of the part increase rapidly, while the proportions and shape remain reasonably constant providing a consistent shape of the end part relative to the original mold. Additional disclosure of such cross-linking and expansion is provided in European Patent 0 802 039 A2, filed on Mar. 25, 1997, and assigned to FINPROJECT™ of Italy.

Some embodiments disclosed herein utilize an EVA as previously described that is known commercially as LEVIREX™ (marketed by FINPROJECT™ of Italy), as well as growth additive such as ENGAGE™ (marketed by DUPONT™), and pigment (which is modified depending upon the desired color of the sexual aid device). It is desirable to create a mixture that exhibits a growth value that encompasses expansion and contraction characteristics, which provide for devices that have an appropriate firmness and size reproductability. This mixture needs further refining when different colored devices are required. This is in part due to the density of the pigment associated with certain colors. Therefore, different growth values are determined for each color required so that accurate and comparable sizes can be manufactured also offering the desired firmness for the various types of devices.

Turning to FIG. 9a, a sexual aid mold 900 in accordance with some embodiments of the present invention is illustrated. Sexual aid mold 900 includes an upper portion 910 and a lower portion 920. Semicircular inlets 950 are formed in both the upper portion 910 and lower portion 920 to allow for inserting molding material into mold 900. One of ordinary skill in the art will recognize that full circle inlets in either the top or the bottom can also be used. Attachment screws 930a, 930b are also included to suspend a sexual aid form from the interior of the mold 900 as more fully described below. As illustrated, upper portion 910 and lower portion 920 come into contact at location 960. In one case, attachment screws 930a, 930b are not threaded, but rather are cylindrical with a clamp attachment groove cut into the end of attachment screws 930 secured to the outer side of upper portion 910. Further, attachment screws 930 include a head region that mates with an internal sexual aid form (see FIG. 9b below). FIG. 9b shows a cut away cross section 901 of sexual aid mold 900. Cross section 901 depicts a sexual aid form 940 suspended within the interior formed by the combination of upper portion 910 and lower portion 920. FIG. 12c shows a top view 902 of the interior of lower portion 920. As illustrated, a sexual aid is formed by inserting molding material through inlets 950 into the molding shape 970. It should be noted that though specific sexual aid devices are illustrated and disclosed in detail, the sexual aid device described hereinabove is contemplated for use with and device designed for sexual/erogenous stimulation of any kind.

While the invention has been described with reference to an exemplary embodiment, it should be understood by those skilled in the art that various changes may be made and equivalents may be substituted for elements thereof without departing from the scope of the invention. In addition, many modifications may be made to adapt a particular situation or substance to the teachings of the invention without departing from the scope thereof. Therefore, it is important that the invention not be limited to the particular embodiment disclosed as the best mode contemplated for carrying out this invention, but that the invention will include all embodiments falling within the scope of the apportioned claims. Moreover, unless specifically stated any use of the terms first, second, etc. do not denote any order or importance, but rather the terms first, second, etc. are used to distinguish one element from another.

What is claimed is:

1. A sexual aid device comprising:
an inner region; and
an outer region, wherein at least said outer region is fabricated of a molded, lofted foam material that is non-porous, wherein said outer region is configured for use with at least one of an orifice, genitalia and erogenous zone of at least one user.

2. The device of claim 1, wherein at least said outer region includes a body shape configured for sexual pleasure.

3. The device of claim 2, wherein said inner region defines a cavity that is open at one end of said body, said cavity being configured to hold an agitator that is associated with a power providing device, wherein said open end of said body is configured to associate with a detachable cap.

4. The device of claim 3, wherein said agitator is motorized, and said power providing device is disposed within said cavity in electric association with said agitator.

5. The device of claim 2, wherein at least said outer region includes a base plate that extends from an end of the body, said base plate being configured to fit snugly against a lower torso of a user, and being of molded, non-releasable formation with said body.

6. The device of claim 1, wherein at least said outer region defines a device body, said device body including a bulbous, conical end, an enlarging region extending from said conical end to a cylindrical portion of said device body, and a decreasing region extending from said cylindrical portion to flange end of said device body, wherein a flange portion of molded, non-releasable formation with said device body extends from said flange end of said device body.

7. The device of claim 6, wherein said conical end includes a first diameter, said cylindrical portion includes a second diameter that is larger than said first diameter, said flange end includes a third diameter that is smaller than said second diameter, and said flange includes a fourth diameter that is greater than said second diameter.

8. The device of claim 1, wherein at least said outer region includes a plurality of ball shaped regions connected via connecting portions, said ball shaped regions and said connecting portions defining a device body of the device.

9. The device of claim 8, wherein said ball shaped regions are of varying size, and pull ring of molded, non-releasable formation with said device body extends from an end of said device body.

10. The device of claim 8, wherein said inner portion defines a string cavity that extends a substantial length of said device body, said string cavity being configured to hold a string component.

11. The device of claim 8, wherein said ball shaped regions define ball cavities configured to hold Ben-Wa-Balls.

12. A sexual aid device comprising:
a piece including an inner region and an outer region, wherein at least said outer region is fabricated of a molded, lofted foam material that is non-porous; and
a base plate being configured to fit snugly against a lower torso of a user, said base plate including an inner base region and an outer base region, wherein at least said outer base region is fabricated of said molded, lofted foam material that is non-porous, and wherein at least said outer base region is of molded, non-releasable formation with at least said outer region of said piece.

13. The device of claim 12, wherein said inner region, said outer region, said inner base region, and said outer base region are all of said molded, lofted foam material that is non-porous, and wherein said inner region, said outer region, said inner base region, and said outer base region are all in molded, non-releasable formation with each other.

14. The device of claim 12, wherein said base plate includes a first strap portion extending from a first lateral end and a second strap portion extending from a second lateral end, said first strap portion and said second strap portion being fabricated of a molded, lofted foam material that is non-porous, said first strap portion and said second strap portion being of molded, non-releasable formation with at least an outer base portion of said base plate, and said first strap portion and said second strap portion being associable with each other to affix said base plate to said user.

15. The device of claim 12, wherein said base plate defines at least two strap cavities configured to associate with a strap, said strap being configured to affix said base plate to said user.

16. The device of claim 1, wherein said lofted foam is a non-toxic material.

17. The device of claim 12, wherein said lofted foam is a non-toxic material.
